(19) United States
(12) Patent Application Publication
(10) Pub. No.: US 2023/0125180 A1
(43) Pub. Date: Apr. 27, 2023

(54) BLOOD PRESSURE MEASUREMENT SYSTEM UTILIZING AUSCULTATORY SIGNAL ACQUISITION

(71) Applicant: WALNUT MEDICAL PRIVATE LIMITED, Sahibzada Ajit Singh Nagar (IN)

(72) Inventors: Mandeep SINGH, Patiala (IN); Jaydeep PANCHAL, Ahmedabad (IN); Amol BHARDWAJ, Panchkula (IN); Siddharth DHAWAN, Ambala City (IN)

(21) Appl. No.: 17/584,641

(22) Filed: Jan. 26, 2022

(30) Foreign Application Priority Data
Oct. 21, 2021 (IN) 202111047949

(51) Int. Cl. A61B 5/0225; A61B 5/00; A61B 5/022; A61B 5/0235

(52) U.S. Cl. CPC A61B 5/0225; A61B 5/6824; A61B 5/02225; A61B 5/6831; A61B 5/0235; A61B 5/02233; A61B 5/7203; A61B 5/746; A61B 5/742; A61B 2562/0204; A61B 2562/0247

(57) ABSTRACT

The present invention provides a system for measurement of blood pressure by detecting the Korotkoff sound. The system comprises an arm band placed on the brachial artery of the user and pressure is applied to the artery by inflating the cuff through an inflation pump provided in a measurement unit, thereby forcing the artery to close. Further, the pressure is reduced by opening at least one valve and by reducing the pressure the Korotkoff sounds are detected using the ausculatory blood pressure monitoring component placed in pneumatic pathway of cuff. The acoustic sound is detected in the audible range of 20 Hz to 10 KHz and also ultrasonic range between 60 KHz to 80 Khz. In this range the signal to noise ratio is maximal. The method for detecting the blood pressure through the present system enhances acoustic signal detection that improves blood pressure measurement accuracy.

### FIELD OF INVENTION

The present invention relates to a blood pressure measurement system. Particularly, the present invention relates to a system and method for improving accuracy by enabling continuous and automatic blood pressure measurement employing real-time acquisition of auscultatory and/or oscillatory signals.

### BACKGROUND OF THE INVENTION

Physiological body parameters play an important role in assessing the physical well-being of people. Such parameters may include heart rate, blood pressure, body temperature, oxygen level, etc. In modern medicine, the monitoring and measurement of blood pressure is crucial in detecting health condition of the people. The measurement of individual's blood pressure helps in preventing various disease arising from hypertension (high blood pressure) and hypotension (low blood pressure).

Blood pressure is measured in two values including systolic and diastolic value. The systolic values indicates the force that blood exerts against the wall of arteries when the heart beats. The diastolic value indicates the force that blood exerts against the wall of arteries when the heart relaxes. The blood pressure may be measured by mainly four methods including invasive, auscultatory oscillometric, and tonometric. The auscultatory and oscillometric methods are the most common methods to detect the blood pressure.

The auscultatory method involves the use of sphygmomanometer and stethoscope. The method further involves positioning an inflatable cuff around the upper arm of a patient. The cuff is further attached to a manometer and is inflated until the brachial artery is completely obstructed. The stethoscope is used to hear blood flow in the brachial artery as the pressure in the cuff is slowly released. As the blood starts to flow in the artery again, the turbulent flow acting against the walls of artery vibrates creating a whooshing/rushing sound known as Korotkoff sound. The korotkoff sound is classified into five phases depending on the quality of sound. Out of the five phases, the first phase of Korotkoff sounds appear at the point when the pressure within the cuff is equivalent to the systolic within the blood vessel. The pressure in the cuff is further released until laminar blood flow is restored. At this stage, there is no sound and represents the fifth phase of Korotkoff sound. The fifth phase is an indicative of diastolic pressure.

The oscillometric method involves monitoring oscillations of pressure in the sphygmomanometer cuff which may result by the oscillations of flow of the blood. The oscillometric method involves a sphygmomanometer cuff; an electronic pressure sensor (transducer) to monitor the oscillations of pressure in the cuff; electronics for automatic interpretation of the oscillations; and automatic inflating and deflating of the sphygmomanometer cuff. The method involves inflating the inflatable cuff positioned on the upper arm of a patient to a predetermined pressure above the patient's estimated systolic pressure. The cuff pressure is further reduced gradually in short period of time with predetermined deflation rate. Further, the transducer converts the cuff pressure to an electrical signal that is further converted to systolic and diastolic values using an algorithm. When blood flow is obstructed and eventually stopped, the cuff pressure will become relatively constant. At this stage, there will be no oscillations. However, when the blood flow is restricted, the cuff pressure will vary with the cyclic expansion and contraction of the brachial artery generating oscillation signals. As the reduction continues, the peak amplitudes of the oscillations will increase from a lower level to a relative maximum, and thereafter will decrease. These amplitudes results in an oscillometric envelope of the patient. The pressure at which the oscillations have a maximum value is an indicative of the mean arterial pressure (MAP).

There are several patent applications comprising a method and system for measuring the blood pressure.

U.S. Pat. No. 5,025,793 discloses a system for measuring blood pressure indirectly by the oscillometric method at the user's index finger. However, this system may not provide accurate results since small amount of blood flows through the finger as compared to the upper arm and may require calibrating the pressure readings equivalent to branchial pressure. Moreover, the oscillometric method is sensitive to body movements due bandwidth of the signals which may be an inconvenient approach for the user.

U.S. Pat. No. 6,805,671B2 discloses a method and device for measuring systolic and diastolic blood pressure and heart rate in an environment with extreme levels of noise and vibrations.

However, in all the above prior arts, the acoustic sensors are usually placed within the pressure cuff to be in close contact with the patient's skin. Such placement of sensors may isolate the acoustic signals from artifacts and extraneous noise which may result in poor detection of acoustic signal leading to inaccurate results.

Therefore, keeping in view the problem associated with the state of the art there is a need of an blood pressure measuring system that provides an accurate measurement of blood pressure and is also provide a portable, convenient and cost-effective.

### OBJECTIVES OF THE INVENTION

The primary objective of the present invention is to provide a blood pressure measurement system measuring the blood pressure of the user based on the detection of Korotkoff sounds.

Another objective of the present invention is to provide a safe, non-invasive system for measuring blood pressure utilizing auscultatory signal acquisition.

Another objective of the present invention is to provide a portable, convenient, cost-effective system for accurate measurement of blood pressure.

Another objective of the present invention is to provide a system with enhanced detection of auscultatory and/or oscillatory signals for efficient measurement of blood pressure.

Yet another objective of the present invention is to provide a system with acoustic sensors placed in pneumatic pathway of pressure cuff for accurate detection of acoustic signals.

Yet another objective of the present invention is to use a low frequency range to avoid disturbances during the detection of signals.

Other objectives and advantages of the present invention will become apparent from the following description taken in connection with the accompanying drawings, wherein, by way of illustration and example, the aspects of the present invention are disclosed.

### SUMMARY OF THE INVENTION

The present invention provides a system and method for measurement of blood pressure in by detecting the Korotkoff sound. The system comprises a measurement unit connected to an arm band by an air hose. The arm band is placed on the brachial artery of the user and pressure is applied to the artery by inflating the cuff through an inflation pump provided in a measurement unit, thereby forcing the artery to close. The measurement unit comprises: an auscultatory blood pressure monitoring component to detect the Korotkoff sounds, a switching means to start the system ON/OFF, a display unit for enabling the user to view levels and battery status, a memory storage unit to store the recorded data into the system and a control system to analyse and evaluate the blood pressure of the user and remove the disturbances from the audio signals. The acoustic sound is detected in the audible range of 20 Hz to 10 KHz and also ultrasonic range between 60 KHz to 80 Khz. In this range the signal to noise ratio is maximal. The method for detecting the blood pressure through the present system enhances acoustic signal detection that improves blood pressure measurement accuracy.

### BRIEF DESCRIPTION OF DRAWINGS

The present invention will be better understood after reading the following detailed description of the presently preferred aspects thereof with reference to the appended drawings, in which the features, other aspects and advantages of certain exemplary embodiments of the invention will be more apparent from the accompanying drawing in which:

FIG. 1 illustrates the schematic view of the blood pressure measurement system of the present invention;

FIG. 2 illustrates the connection diagram of the control system;

FIG. 3 illustrates the flow diagram depicting the method of working a blood pressure measurement system.

Reference Numeral incorporated in the drawings are discussed herein:

| S. No. | Reference Numeral | Components |
|---|---|---|
| 1. | 100a | a blood pressure measurement system |
| 2. | 100 | measurement unit |
| 3. | 101 | arm band |
| 4. | 102 | air hose |
| 5. | 103 | acoustic sensor |
| 6. | 104 | ON/OF switch |
| 7. | 105 | display unit |
| 8. | 106 | inflation pump |
| 9. | 107 | bleed valve |
| 10. | 108 | control system |
| 11. | 109 | pressure sensor |
| 12. | 110 | storage device |
| 13. | 111 | battery unit |
| 14. | 112 | alert unit |
| 15. | 113 | Driver Circuit |
| 16. | 114 | Low-Dropout regulator (LDO) |

### DETAILED DESCRIPTION OF THE INVENTION

The following detailed description and embodiments set forth herein below are merely exemplary out of the wide variety and arrangement of instructions which can be employed with the present invention. The present invention may be embodied in other specific forms without departing from the spirit or essential characteristics thereof. All the features disclosed in this specification may be replaced by similar other or alternative features performing similar or same or equivalent purposes. Thus, unless expressly stated otherwise, they all are within the scope of the present invention.

Accordingly, those of ordinary skill in the art will recognize that various changes and modifications of the embodiments described herein can be made without departing from the scope of the invention. In addition, descriptions of well-known functions and constructions are omitted for clarity and conciseness.

The terms and words used in the following description and claims are not limited to the bibliographical meanings but are merely used to enable a clear and consistent understanding of the invention. Accordingly, it should be apparent to those skilled in the art that the following description of exemplary embodiments of the present invention are provided for illustration purpose only and not for the purpose of limiting the invention.

It is to be understood that the singular forms “a”, “an” and “the” include plural referents unless the context clearly dictates otherwise.

It should be emphasized that the term “comprises/comprising” when used in this specification is taken to specify the presence of stated features, integers, steps, or components but does not preclude the presence or addition of one or more other features, integers, steps, components, or groups thereof.

The present invention relates to a digital blood pressure measurement system (100a) that accurately measures the blood pressure of the patient/user utilizing the ausculatory method, oscillometric method and/or a combination thereof. The system (100a) provides a continuous and measurement of blood pressure gradient to any operator for e.g., doctors, caretaker, nurse, medical practitioner etc. to assess the pressure in the arteries as the user's heart pumps.

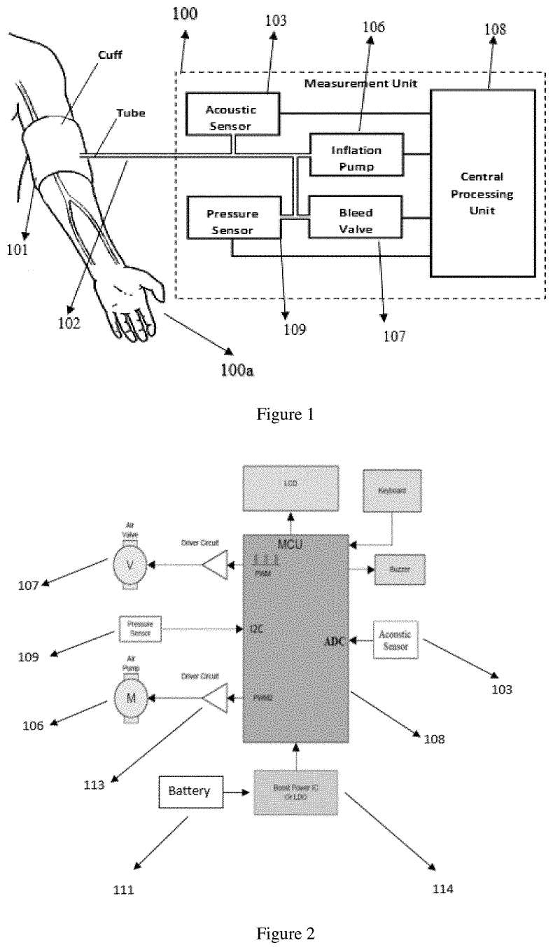

In the present invention FIG. 1 illustrates a schematic view of the digital blood pressure measurement system (100a) comprising a measurement unit (100) removably connected with an arm band (101) through the air hose (102). The arm band (101) is configured to be positioned on the patient's arm/brachial artery. The measurement unit (100) of the present invention comprises an ON/OF switch (104) for powering on and off the system; a display unit (105) connected with a screen to view stored and/or recorded blood pressure data; an inflation pump (106) providing air pressure to the arm band (101); a bleed valve (107) for inflating and deflating the arm band (101); a control system (108) embedded with software module for controlling the operation of digital blood pressure measurement system (100a); an acoustic sensor (103) for capturing vibrational noises/Korotkoff sounds; a pressure sensor (109) for measuring the auscultatory blood pressure of the patient; a storage device (110) configured to storage the blood pressure data of the user; a battery unit (111) providing power supply to the digital blood pressure measurement system (100a); and an alert unit (112) to alarm the user about unusual blood pressure reading. The following components of the measurement unit (100) are discussed herein in detail:

(a) ON/OFF Switch (104): The switch mounted on the measurement unit (100) enables the user to power ON and OFF the operation of the machine. The switch is configured to control connection or disconnection of all the components of the system (100a).

(b) Digital Display Unit (105): The display unit (105) provided in the measurement unit (100) enables the user to view the stored and/or recorded data on the screen of the display unit (105). The display unit (105) includes a display screen such as, but not limited to, a liquid crystal display (LCD), light emitting diodes (LEDs), or organic light emitting diodes (OLEDs). The display screen may be a touch screen display. The screen of the digital display unit (105) enables the user to view numerical values for the blood pressure measurements, displaying operational instructions entered by a user, indicating the acceptable range for types of blood pressure measurement values, alarm in case of unusual blood pressure reading etc. The display unit (105) displays the instructions selected by the user i.e., a keyboard, a key pad, scrolling buttons, soft buttons whose functions change depending on the menu presented to the user.

(c) Inflation Pump (106): The inflation pump (106) provides air pressure to the arm band (101) by means of the air hose (102). In an alternate embodiment, the air pressure to the arm band (101) can be provided with any source of compressed gas which can be controlled by the command given by the control system (108) so as to provide an appropriate amount of gas pressure to the arm band (101).

(d) Bleed Valve (107): The bleed valve (107) helps in controlling the inflation and deflation of the arm band (101).

(e) Control System (108): FIG. 2 illustrates the connection of various electronic components with the control system. The control system (108) incorporates a controlling device such as, but not limited to, a microcontroller, a microprocessor, Arduino, etc. configured with a software module. The control system (108) measures, monitors, evaluates, and displays the blood pressure of the patient/user. The control system (108) manages bleed valve (107) and inflation pump (106) with pulse width modulation (PWM). Further, the PWM signal provided by the control system does not have enough power to run the motor/valve. A driver circuit (113) is configured to increase the power of PWM signal suitable for driving motor/valve. Furthermore, voltage converters like Low-Dropout regulator (LDO) (114) or Boost converter are configured to convert battery voltage into operating voltage of control system. The software module enables a user to control the operation of the system (100a). The input provided by the user into the system is translated into machine language understandable by the control system (108) so as to operate the arm band (101), the inflation pump (106) for inflating or deflating the air pressure provided to arm band (101) and the acoustic sensor (103) for recording the Korotkoff sounds. The software module helps in converting waveforms corresponding to sounds recorded by the acoustic sensor and the pressure sensor from the arm band (101) into values for evaluating systolic and diastolic arterial blood pressure.

(f) Acoustic sensor (103): It is an ausculatory blood pressure monitoring component for capturing vibrational noises/Korotkoff sounds, which are produced by releasing air pressure from an arm band (101) placed on the brachial artery. The acoustic sensor connected with the control system (108). The software module configured in the controlling device facilitates filtering of the audio signals received from the acoustic sensor (103) so as to remove the noise or any disturbances from the audio signals. It also detect various types of Korotkoff sounds, such as the turbulences created from initial blood flow and the change in sound indicating the transition to laminar blood flow. In an exemplary embodiment the acoustic sensor (103) incorporates a high-fidelity microphone in a pneumatic pathway that enables the system (100a) to measure and record the Korotkoff sound within the audible range of 20 Hz to 10 KHz and ultrasonic range of 60 KHz to 80 KHz.

(g) Pressure Sensor (109): A pressure sensor (transducer) (109) is configured to control and manage the fluctuations in air pressure provided to the arm band (101) during the deflation of the air/gas. The pressure sensor transmits the detected fluctuation via signal to the control system (108) that analyses the fluctuation in blood pressure through the software module so as to obtain the blood pressure measurements. The pressure sensor (109) enables the inflation pump (106) to supply the required air into the arm band (101) so as to enable the ausculatory blood pressure monitoring component to detect the Korotkoff sounds on deflation of air from the arm band (101). The pressure sensor is embedded within the measurement unit (100) that enables the hose to detect the oscillations in pressure at the arm band (101) so as to measure the blood pressure of the user.

(j) Storage device (110): A storage device (110) is coupled to the control system (108) and configured to receive the blood pressure measurement data to store them in the memory for future reference of the user. The storage device may be a non-volatile memory storage including but not limited to a read-only memory (ROM), EPROM (erasable programmable ROM) and EEPROM (electrically erasable programmable ROM) etc.

(k) Battery Unit (111): A battery unit (111) embedded in the measurement unit (100) provides power to the system. The battery may be selected from “AA” alkaline or manganese batteries to supply power to the system.

(l) Alert Unit (112): An alert unit (112) such as, but not limited to, a buzzer or an alarm unit that alerts the user when one or more blood pressure measurements acquired by the arm band (101) and/or the auscultatory blood pressure monitoring component are not within a normal range.

Further, the measurement unit (100) is also configured to process the recorded measurements to derive values for systolic and diastolic blood pressure and display these values along with the original measurements of the detected oscillometric blood pressure measurements. The measurement unit (100) enables the user to store and display multiple blood pressure measurement values on the display unit (105), associated each with a particular time, date, and/or patient's name.

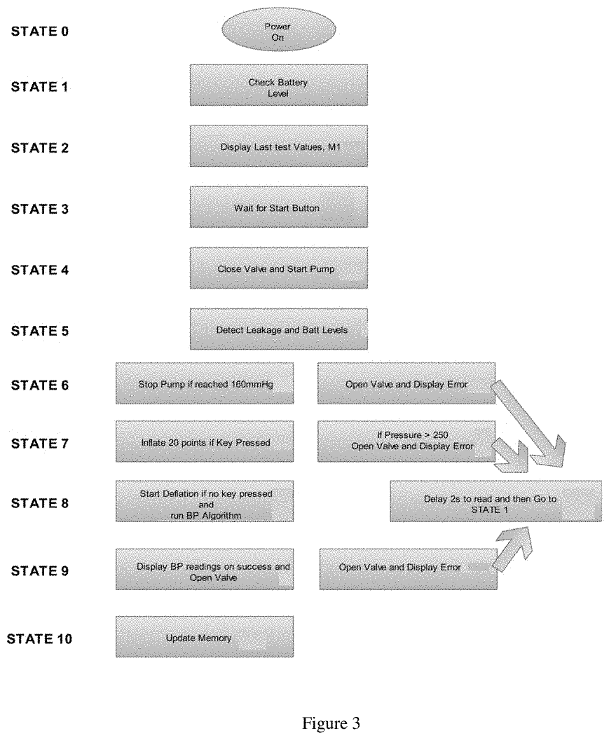

In a preferred embodiment of the present invention, FIG. 3 illustrates the method for using a digital blood pressure measurement system (100a) using the following steps:
a.) wrapping the arm band (101) around the brachial artery of the user;
b.) powering ON the system (100a);
c.) checking the battery level of the system (100a) displayed on the display unit (105);
d.) displaying the last test values related to blood pressure of the user on the display unit (105);
e.) pressing the start button to initiate measurement of blood pressure of the user;
f.) closing the bleed valve (107) and supplying the air pressure to the arm band (101) through the inflation pump (106);
g.) detecting leakage from the air hose (102) and checking the battery unit (111) status;
h.) halting the inflation pump (106) on reaching inflation to a predetermined level for e.g., 160 mmHg;
i.) changing inflation threshold in case start button is pressed during the machine operation;
j.) obtaining pressure oscillation and ausculatory signal on deflation of the arm band (101) through the pressure sensor (transducer) (109);
k.) analysing pressure oscillation measurements and ausculatory signals through the software module;
l.) displaying blood pressure measurements on the display unit (105);
m.) opening the bleed valve (107) to release the pressure from the arm band (101);
n.) detecting Korotkoff sound produced on deflation by the acoustic sensor (103); and
o.) recording and/or updating blood pressure measurements to the storage device (110).

In a preferred embodiment of the present invention, FIG. 3 further illustrates the method for using a digital blood pressure measurement system (100a) in case of detecting error while taking blood pressure readings, the method comprises the following steps:
i. wrapping the arm band (101) around the brachial artery of the user;
ii. powering ON the system (100a);
iii. checking the battery level of the system (100a) displayed on the display unit (105);
iv. displaying the last test values related to blood pressure of the user on the display unit (105);
v. pressing the start button to initiate measurement of blood pressure of the user;
vi. closing the bleed valve (107) and supplying the air pressure to the arm band (101) through the inflation pump (106);
vii. detecting leakage from the air hose (102) and checking the battery unit (111) status;
viii. detecting pressure less than 250 mmHg;
ix. opening bleed valve (107) to release the pressure and displaying error on the display unit;
x. providing delay of at least 2 second to reconfigure the system; and
xi. analyse the detected error and restart the system (100a).

The advantages of the system (100a) and method of the present invention includes:
The system (100a) as per the present invention detects the acoustic sound in the audible range of 20 Hz to 10 KHz and also ultrasonic range 60 KHz to 80 KHz so as to maximise the signal to noise ratio, as per experiments;
The method of the present invention enhances the acoustic signal detection thereby improving the blood pressure measurement accuracy;
The system (100a) enables the operator to assess the blood pressure of the patient/user and thereby provide adequate treatment in case of unusual blood pressure reading;
The system (100a) ensures accurate detection of acoustic signals and removes the noise disturbances by placing the acoustic sensor (103) within the measuring unit in a pneumatic pathway of the arm band (101);
The system (100a) enables selection of frequency range for detecting the Korotkoff sounds that avoids disturbances during the detection of signals;
The whole system (100a) and method thereof, is independent of any assistance from any other people and is highly accurate;
The system (100a) as per present invention eliminates the need to remember the blood pressure measurements and removes the limitation to manually record the previously measured blood pressure.
The present invention provides complete solution to the user by measuring blood pressure gradient in a variety of medical facilities such as, but not limited to, hospital ward, operating room, intensive care unit, recovery, and the emergency room.

While this invention has been described in connection with what is presently considered to be the most practical and preferred embodiment, it is to be understood that the invention is not limited to the disclosed embodiments, but, on the contrary, is intended to cover various modifications and equivalent arrangements included within the scope of the appended claims.

We claim:

1. A blood pressure measurement system (100a), comprising:
a) a measurement unit (100) connected to an arm band (101);
b) the arm band (101) positioned on brachial artery of a user;
c) an air hose (102) removably attached between the arm band (101) and the measurement unit (100);
wherein,
i. an acoustic sensor (103) configured within the measurement unit (100) is positioned in the pneumatic pathway of the arm band (101); and
ii. the acoustic sensor (103) configured to capture Korotkoff sounds from brachial artery of the user with audible range of 20 Hz to 10 KHz and ultrasonic range of 60 KHz to 80 KHz.

2. The system (100a) as claimed in claim 1, wherein the measurement unit (100) comprises of:
i. an ON/OF switch (104) for powering on and off the system;
ii. a display unit (105) connected with a screen to view stored and/or recorded blood pressure data;
iii. an inflation pump (106) providing air pressure to the arm band (101);
iv. a bleed valve (107) for inflating and deflating the arm band (101);
v. a control system (108) embedded with software module for controlling the operation of digital blood pressure measurement system (100a);
vi. the acoustic sensor (103) for capturing vibrational noises/Korotkoff sounds;
vii. a pressure sensor (109) for measuring the auscultatory blood pressure of the patient;
viii. a storage device (110) configured to storage the blood pressure data of the user;
ix. a battery unit (111) providing power supply to the digital blood pressure measurement system (100a); and
x. an alert unit (112) to alarm the user about unusual blood pressure reading.

3. The system (100a) as claimed in claim 1, wherein the arm band (101) is connected to the inflation pump (106) to supply air pressure to the arm band (101).

4. The system (100a) as claimed in claim 1, wherein the arm band (101) inflates or deflates by managing pressure through bleed valve (107).

5. The system (100a) as claimed in claim 1, wherein the software module filters the audio signals received from the acoustic sensor (103) to remove the noise from the recorded audio signals.

6. The system (100a) as claimed in claim 1, wherein the alert unit (112) signals the user on detection of unusual blood pressure reading.

7. The system (100a) as claimed in claim 1, wherein the driver circuit (113) is configured to increase the power of pulse width modulation (PWM) signal suitable for driving motor/valve.

8. The system (100a) as claimed in claim 1, wherein the Low-Dropout regulator (LDO) (114) is configured to convert battery voltage into operating voltage of control system (108).

9. A method for monitoring blood pressure via the blood pressure measurement system (100a) as claimed in claim 1, comprising the following steps:
Step 1. wrapping the arm band (101) around the brachial artery of the user;
Step 2. powering ON the system (100a);
Step 3. checking the battery level of the system (100a) displayed on the display unit (105);
Step 4. displaying the last test values related to blood pressure of the user on the display unit (105);
Step 5. pressing the start button to initiate measurement of blood pressure of the user;
Step 6. closing the bleed valve (107) and supplying the air pressure to the arm band (101) through the inflation pump (106);
Step 7. detecting leakage from the air hose (102) and checking the battery unit (111) status;
Step 8. halting the inflation pump (106) on reaching inflation to a predetermined level for e.g., 160 mmHg;
Step 9. changing inflation threshold in case start button is pressed during the machine operation;
Step 10. obtaining pressure oscillation and auscultatory signal on deflation of the arm band (101) through the transducer (109);
Step 11. analysing pressure oscillation measurements and auscultatory signals through the software module;
Step 12. displaying blood pressure measurements on the display unit (105);
Step 13. opening the bleed valve (107) to release the pressure from the arm band (101);
Step 14. detecting Korotkoff sound produced on deflation by the acoustic sensor (103); and
Step 15. recording and/or updating blood pressure measurements to the storage device (110).

10. A method for detecting error while taking blood pressure readings via the blood pressure measurement system (100a) as claimed in claim 1, comprising the following steps:
i. wrapping the arm band (101) around the brachial artery of the user;
ii. powering ON the system (100a);
iii. checking the battery level of the system (100a) displayed on the display unit (105);
iv. displaying the last test values related to blood pressure of the user on the display unit (105);
v. pressing the start button to initiate measurement of blood pressure of the user;
vi. closing the bleed valve (107) and supplying the air pressure to the arm band (101) through the inflation pump (106);
vii. detecting leakage from the air hose (102) and checking the battery unit (111) status;
viii. detecting pressure less than 250 mmHg;
ix. opening bleed valve (107) to release the pressure and displaying error on the display unit;
x. providing delay of at least 2 second to reconfigure the system; and
xi. analyse the detected error and restart the system (100a).
